SEQUENCE LISTING (13 sequences)

SEQ ID NO: 1 - DNA, 18 bp, synthetic construct
  source 1..18: mol_type = other DNA
atggatgacagttgcgcg

SEQ ID NO: 2 - DNA, 25 bp, synthetic construct
  source 1..25: mol_type = other DNA
ctatttctttccattgctggaaaag

SEQ ID NO: 3 - DNA, 2586 bp, Triticum urartu
  source 1..2586: mol_type = other DNA
atggatgacagttgcgcggtgtgcgcggacgcgctcgaatgggtggcttacgggccgtgcgggcaccgcgaggtgtgttcaacctgcgtcgtccgccttcgcttcgtactcgaggattctctatgctgcatctgtaagacggattgcccctccgtcttcgtcacaaaggccatgggagattacacaaaagtgatctcggatttctctgttttgcccactgaggcaaatgagggaaatgtgggggagtactggtaccatgaggatacaaaggcatactttgatgatgctgatcattataagatgataagggcgatgtgccgactttcttgtagtgtatgtgacaaagctgaggaccaggttggtcaggcagcacaagcaaagcgccgaagcaggttcaagagcattgatcagctaaagggacatttgttccatcagcataggttatacatgtgtaatctttgcttggaggggagaaaggtattcatttgtgaacagaagctttatacaagggcacagttagctcagcatacaaaaactggtgactctgaggtggatggctctgagattgaacgcagtggttttgcaggacacccagtgtgtgaattttgtaaatatccattatatggagataatgagctttacacacatatgtccagagaacactattcgtgccacatatgtcaaaggcagcatcctgggcagtatgattatttccggaactatgatgatttagagatgcattttcgtaaagatcatttcctctgtgaagatgatgcatgtttggccaagaaatttgttgtcttccagagtgacgcagagatcaagagacataatgctatggagcatggtgggcggatgtctcgtgcccagaggaatgcggcacttcagatacctaccagttttatataccaaaggaatgagcaagatcaaaggcgtggcagaggtaggggtcgtaatgctcaccatgacagacctgacagggatttctcattacctgtgcgggatggcagtgcaactgcagaccatggccttggaagtcgagttgatagtgttgcagggcctttccagtcattaagtgtcagttccagttctggtcgaacagaaactggtcgaagcttagggaatggtcgcttgctcgagcagttgtcttttcctccacttcaagatcaggatattcctgatgccaggatggatgctgttccctatgaaacctcgtttcctcccgtctcagagcagcaatcaaggtatgcgctggctcttaatcagagctcaaggggttctgcgaggcttggtgatgaatcattattccctccattgcctgggtcaagtaacaagggttctgcttcaacacaacaggggttgcaaagtcttgctaagaacacacttgcatcaaggctgcaacaacgtagtaagggcaccgtgaaggtactatattctgctcagtctcaaacagctgaaaatcctgagattgtacctcatgtttccacttccacccagacatggcctacacctgagcaggggttacatctctctggttcttctcaactttggattgtaactcaatcaacaagagaaaatgggctcatgccatctgcctccagcggttcagcatggaattccagagcttcaaacaagatgaagcactctatttcgacccctaattttgtttctggtgggtcctctgcccaggcatcaagtacagcttatggcaataaaaagcaactgccaccgcaaagcagccaacctttgcctgttgtggaggatgttcggcaagcaaacaaatcccttgtggaaagaatgcgtgttgcattaggaatggatgaggataggttctctgcgttcaaagaaattgctagtgaataccgtcaaggtgtcattgatacttcagagtatctctcgtatgtggagcagtttggtatatcacatcttgttcctgaaatggctaagttgttgcctgatcctctgaagcagatggaacttgctgatgcctattacaccaacatgcgttttagaagtctccaagaaaacggcggttgtggaaccattactgtgaaagagaacaagcgtagaaataaggggaagggaaaaacacctgatgcagaaacagctcctgctaaggatgcaagtgaatcgctagctgatagctttatggacactgtaaggaagcttcagttgaacaacaaggctcaagaaggagaggctgcagtgctttcgaaggatgggtatcgatcttccaaggaaaaaatcccactatcagctggagggtcatcctgtggtacaaatatgggtctagacggtgatccagttgccatttcaaaggcgtctggcactagtaggtatgtgggcaagggtggaggaagcaccggtagcagcagcagtaacaacaaacaatcgaagaagacatcaaagtttctcagagctcggttgggtgacaactcattggctacacttgattttagtcatcctgatgtgagccctgaacgacctgtaagggagacacaagtcctgcaaactgggttgcctgtgcgaagtgtttggaagaatggtgcagcacagaagctcttttccagcaatggaaagaaatag

SEQ ID NO: 4 - protein, 861 aa, Triticum urartu
MDDSCAVCADALEWVAYGPCGHREVCSTCVVRLRFVLEDSLCCICKTDCPSVFVTKAMGDYTKVISDFSVLPTEANEGNVGEYWYHEDTKAYFDDADHYKMIRAMCRLSCSVCDKAEDQVGQAAQAKRRSRFKSIDQLKGHLFHQHRLYMCNLCLEGRKVFICEQKLYTRAQLAQHTKTGDSEVDGSEIERSGFAGHPVCEFCKYPLYGDNELYTHMSREHYSCHICQRQHPGQYDYFRNYDDLEMHFRKDHFLCEDDACLAKKFVVFQSDAEIKRHNAMEHGGRMSRAQRNAALQIPTSFIYQRNEQDQRRGRGRGRNAHHDRPDRDFSLPVRDGSATADHGLGSRVDSVAGPFQSLSVSSSSGRTETGRSLGNGRLLEQLSFPPLQDQDIPDARMDAVPYETSFPPVSEQQSRYALALNQSSRGSARLGDESLFPPLPGSSNKGSASTQQGLQSLAKNTLASRLQQRSKGTVKVLYSAQSQTAENPEIVPHVSTSTQTWPTPEQGLHLSGSSQLWIVTQSTRENGLMPSASSGSAWNSRASNKMKHSISTPNFVSGGSSAQASSTAYGNKKQLPPQSSQPLPVVEDVRQANKSLVERMRVALGMDEDRFSAFKEIASEYRQGVIDTSEYLSYVEQFGISHLVPEMAKLLPDPLKQMELADAYYTNMRFRSLQENGGCGTITVKENKRRNKGKGKTPDAETAPAKDASESLADSFMDTVRKLQLNNKAQEGEAAVLSKDGYRSSKEKIPLSAGGSSCGTNMGLDGDPVAISKASGTSRYVGKGGGSTGSSSSNNKQSKKTSKFLRARLGDNSLATLDFSHPDVSPERPVRETQVLQTGLPVRSVWKNGAAQKLFSSNGKK

SEQ ID NO: 5 - DNA, 46 bp, synthetic construct
  source 1..46: mol_type = other DNA
gattcttcttccgttgctagcgactttcttgtagtgtatgtgacaa

SEQ ID NO: 6 - DNA, 46 bp, synthetic construct
  source 1..46: mol_type = other DNA
ttttttttttttttagctagcaagattacacatgtataacctatgc

SEQ ID NO: 7 - DNA, 140 bp, Triticum urartu
  source 1..140: mol_type = other DNA
gactttcttgtagtgtatgtgacaaagctgaggaccaggttggtcaggcagcacaagcaaagcgccgaagcaggttcaagagcattgatcagctaaagggacatttgttccatcagcataggttatacatgtgtaatctt

SEQ ID NO: 8 - DNA, 24 bp, synthetic construct
  source 1..24: mol_type = other DNA
tggtgtcatcaagcctggtatggt

SEQ ID NO: 9 - DNA, 24 bp, synthetic construct
  source 1..24: mol_type = other DNA
actcatggtgcatctcaacggact

SEQ ID NO: 10 - DNA, 24 bp, synthetic construct
  source 1..24: mol_type = other DNA
ttcgccgtccgtgatatgagacaa

SEQ ID NO: 11 - DNA, 24 bp, synthetic construct
  source 1..24: mol_type = other DNA
atgcgtatcatggtggtggagtga

SEQ ID NO: 12 - DNA, 20 bp, synthetic construct
  source 1..20: mol_type = other DNA
tttcaaaggcgtctggcact

SEQ ID NO: 13 - DNA, 21 bp, synthetic construct
  source 1..21: mol_type = other DNA
tgtagccaatgagttgtcacc

(19) United States
(12) Patent Application Publication
(10) Pub. No.: US 2026/0185116 A1
(43) Pub. Date: Jul. 2, 2026

(54) WHEAT E3 UBIQUITIN LIGASE GENE RFEL1 AND APPLICATION IN WHEAT DISEASE RESISTANCE THEREOF

(71) Applicant: Henan Agricultural University, Zhengzhou City (CN)

(72) Inventors: Huan WANG, Zhengzhou City (CN); Daowen WANG, Zhengzhou City (CN); Jinyan LI, Zhengzhou City (CN); Ximei MA, Zhengzhou City (CN); Kunpu ZHANG, Zhengzhou City (CN); Jianhui ZHAO, Zhengzhou City (CN); Ziming ZHANG, Zhengzhou City (CN)

(21) Appl. No.: 19/433,161

(22) Filed: Dec. 26, 2025

(30) Foreign Application Priority Data
Dec. 30, 2024 (CN) 202411962799.2

(51) Int. Cl. C12N 15/82; C07K 14/415; C12N 9/10

(52) U.S. Cl. CPC C12N 15/8282; C07K 14/415; C12N 9/104; C12Y 203/02

(57) ABSTRACT

A wheat E3 ubiquitin ligase gene RING-finger E3 ligase 1 (RFEL1) and its application in wheat disease resistance. The nucleotide sequence of the wheat E3 ubiquitin ligase gene RFEL1 is as shown in SEQ ID NO. 3. The present disclosure further provides an application of the wheat E3 ubiquitin ligase gene RFEL1 or related biological materials thereof, including any one of the following applications: A1, enhancing wheat resistance to stripe rust; A2, enhancing wheat resistance to powdery mildew; and A3, enhancing wheat resistance to leaf rust.

### CROSS-REFERENCE TO RELATED APPLICATIONS

This application claims priority to Chinese Patent Application No. 202411962799.2, filed on Dec. 30, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Dec. 23, 2025
Byte size: 16,153

### TECHNICAL FIELD

The present disclosure relates to the technical field of plant genetic engineering, and in particular to a wheat E3 ubiquitin ligase gene RING-finger E3 ligase 1 (RFEL1) and its application in wheat disease resistance.

### BACKGROUND

Wheat is one of the most important food crops in the world and in China. Its global planting area is second only to that of corn and rice, providing staple food for approximately 35 percent (%) of the world's population. Ensuring the safe and efficient production of wheat is crucial for guaranteeing global food and nutritional security. However, the existence and continuous prevalence of plant diseases pose serious challenges to the safe production of wheat. During the growth of wheat, several severe fungal diseases persistently and frequently threaten its yield and quality. These include stripe rust caused by Puccinia striiformis f. sp. tritici, leaf rust caused by Puccinia triticina, powdery mildew caused by Blumeria graminis f. sp. tritici, as well as Fusarium head blight and crown rot caused by various Fusarium species.

Cultivating wheat varieties with resistance is a very effective, economically saving, and environmentally friendly measure for controlling stripe rust. However, due to continuous mutation of the stripe rust fungus and the emergence of new physiological races, the stripe rust resistance of disease-resistant varieties under long-term and monoculture cultivation may be overcome. With the intensification of global climate change, the epidemic areas of stripe rust are continuously expanding, resulting in increasing losses. To efficiently breed wheat varieties with stripe rust resistance, it is necessary to discover more wheat genes conferring resistance to stripe rust and to fully understand the molecular mechanisms by which they function. This will provide genetic resources and a theoretical foundation for better breeding wheat varieties with durable and stable resistance to stripe rust.

### SUMMARY

The objective of the present disclosure is to provide a wheat E3 ubiquitin ligase gene RING-finger E3 ligase 1 (RFEL1) and its application in wheat disease resistance, so as to solve the problems existing in the aforementioned prior art. The wheat E3 ubiquitin ligase gene RFEL1 may be used in wheat breeding for broad-spectrum resistance.

To achieve the above objective, the present disclosure provides the following schemes:
technical scheme 1: a wheat E3 ubiquitin ligase gene RFEL1, where the nucleotide sequence of the wheat E3 ubiquitin ligase gene RFEL1 is as shown in SEQ ID NO. 3.
Technical scheme 2: an application of the wheat E3 ubiquitin ligase gene RFEL1 or related biological materials thereof, including any one of the following applications:
A1, enhancing wheat resistance to stripe rust;
A2, enhancing wheat resistance to powdery mildew; and
A3, enhancing wheat resistance to leaf rust.

In an embodiment, the related biological materials include any one of the following B1 to B5:
B1, a protein encoded by the wheat E3 ubiquitin ligase gene RFEL1;
B2, an expression cassette including the wheat E3 ubiquitin ligase gene RFEL1;
B3, a recombinant vector including the wheat E3 ubiquitin ligase gene RFEL1;
B4, a recombinant microorganism including the wheat E3 ubiquitin ligase gene RFEL1; and
B5, a transgenic material including the wheat E3 ubiquitin ligase gene RFEL1.

In an embodiment, the amino acid sequence of the protein encoded by the wheat E3 ubiquitin ligase gene RFEL1 is as shown in SEQ ID NO. 4.

The recombinant vector includes the wheat E3 ubiquitin ligase gene RFEL1 having a sequence as shown in SEQ ID NO. 3; and the recombinant microorganism includes the wheat E3 ubiquitin ligase gene RFEL1 having a sequence as shown in SEQ ID NO. 3.

Technical scheme 3: an application of a functional product that upregulates the expression level of the wheat E3 ubiquitin ligase gene RFEL1 in enhancing wheat resistance to stripe rust, powdery mildew, and leaf rust, where the functional product is a gene RFEL1 overexpression vector.

Technical scheme 4: a method for enhancing wheat resistance to stripe rust, powdery mildew, and leaf rust, including the following steps:
step 1, constructing an overexpression vector using the wheat E3 ubiquitin ligase gene RFEL1 as a target gene;
step 2, transforming the constructed overexpression vector into wheat; and
step 3, screening to obtain positive transgenic wheat.

The present disclosure discloses the following technical effects.

In the present disclosure, transcriptome sequencing analysis is performed on samples from Triticum urartu accession PI428322 before and after inoculation with wheat stripe rust fungus, and an E3 ubiquitin ligase gene RFEL1 is identified to be up-regulated upon induction by the stripe rust fungus. Preliminary verification using virus-induced gene silencing (VIGS) technology indicates that the RFEL1 gene positively regulates wheat resistance to stripe rust. Triticum urartu is a diploid wild einkorn wheat and is the ancestral species of the A genome of tetraploid and hexaploid wheat. Triticum urartu possesses abundant germplasm resources in nature, which may serve as gene resource for discovering stripe rust resistance genes and be applied in common wheat breeding. Furthermore, transgenic technology is employed to introduce the gene into the common hexaploid wheat cultivar Fielder to verify its role in positively regulating wheat resistance to stripe rust. The results of stripe rust resistance identification show that the RFEL1 overexpression lines exhibits resistance to different stripe rust physiological races CYR17, CYR32, CYR33, and CYR34. Further resistance assays demonstrate that the RFEL1 overexpression lines also possess resistance to both powdery mildew and leaf rust. The E3 ubiquitin ligase gene RFEL1 may be used in wheat breeding for broad-spectrum resistance and possesses significant application value. The present disclosure identifies a broad-spectrum disease resistance gene, providing theoretical guidance and genetic gene resources for wheat disease resistance breeding.

### BRIEF DESCRIPTION OF THE DRAWINGS

To illustrate the embodiments of the present disclosure or the technical schemes in the prior art more clearly, the following briefly introduces the drawings required for describing the embodiments. Obviously, the drawings in the following description are merely some embodiments of the present disclosure. For those of ordinary skill in the art, other drawings may be derived from these drawings without creative effort.

FIG. 1A, FIG. 1B, and FIG. 1C show that gene silencing of RING-finger E3 ligase 1 (RFEL1) induced by barley stripe mosaic virus (BSMV) in Triticum urartu material PI428322 may reduce the resistance of this material to stripe rust fungus CYR34, where: FIG. 1A shows the relative expression level of the RFEL1 gene in BSMV: green fluorescent protein (GFP) and BSMV:RFEL1 plants; FIG. 1B shows that the resistance of the resistant material PI428322 to stripe rust fungus CYR34 is weakened after silencing RFEL1; and FIG. 1C shows that the area occupied by stripe rust fungus spores on the leaf surface of BSMV:RFEL1 plants is significantly higher than that on BSMV:GFP plants.

FIG. 2A, FIG. 2B, and FIG. 2C show that stable transformation of the susceptible common wheat cultivar Fielder with the RFEL1 gene, and the RFEL1 overexpression plants exhibit enhanced resistance to stripe rust fungus CYR34, where: FIG. 2A shows the expression level of the RFEL1 gene in positive RFEL1 overexpression plants and wild-type Fielder as identified by quantitative reverse transcription polymerase chain reaction (qRT-PCR); FIG. 2B shows that overexpression of RFEL1 enhances the resistance of common wheat Fielder to stripe rust fungus CYR34; and FIG. 2C shows that the biomass of the stripe rust fungus in infected leaves of positive RFEL1 overexpression plants is significantly lower than that in wild-type Fielder.

FIG. 3 shows that RFEL1 overexpression plants exhibit enhanced wheat resistance to stripe rust fungi CYR17, CYR32, and CYR33.

FIG. 4A and FIG. 4B show that RFEL1 overexpression plants exhibit enhanced wheat resistance to powdery mildew fungus E09, where: FIG. 4A shows the cell death and the accumulation of hydrogen peroxide (H₂O₂) in RFEL1 overexpression plants and wild-type Fielder plants at 48 hours after inoculation with powdery mildew fungus E09; and FIG. 4B shows that overexpression of RFEL1 enhances the resistance of common wheat Fielder to powdery mildew fungus E09.

FIG. 5A and FIG. 5B show that RFEL1 overexpression plants exhibit enhanced wheat resistance to leaf rust fungus Pt23a, where: FIG. 5A shows the phenotypes of RFEL1 overexpression plants and wild-type Fielder plants at 14 days after inoculation with leaf rust fungus Pt23a; and FIG. 5B shows that the biomass of the leaf rust fungus in infected leaves of positive RFEL1 overexpression plants is significantly lower than that in wild-type Fielder.

### DETAILED DESCRIPTION OF THE EMBODIMENTS

The various exemplary embodiments of the present disclosure are described in detail below. This detailed description should not be construed as limiting the disclosure, but rather should be understood as providing a more detailed description of certain aspects, characteristics, and embodiments of the present disclosure.

It should be understood that the terms used herein are only for describing particular embodiments and are not intended to limit the present disclosure. Furthermore, for numerical ranges recited in the present disclosure, it should be understood that each intermediate value between the upper and lower limits of the range is also specifically disclosed. Each smaller range between any stated value or intermediate value within a stated range and any other stated or intermediate value within The range is also encompassed within the present disclosure. The upper and lower limits of these smaller ranges may independently be included or excluded in the range.

Unless otherwise defined, all technical and scientific terms used herein have the same meaning as commonly understood by one of ordinary skill in the art to which this disclosure belongs. Although only optional methods and materials are described herein, any methods and materials similar or equivalent to those described may also be used in the practice or testing of the present disclosure. All publications mentioned in this specification are incorporated by reference to disclose and describe the methods and/or materials related to the publications. In case of conflict with any incorporated publication, the content of this specification shall prevail.

Various modifications and changes to the specific embodiments described in the specification of the present disclosure may be made without departing from the scope or spirit of the present disclosure, which will be apparent to those skilled in the art. Other embodiments obtained from the description of the present disclosure will be apparent to those skilled in the art. The specification and embodiments of the present disclosure are exemplary only.

Concerning the terms “comprising”, “including”, “having”, “containing”, etc., used herein, they are open-ended terms, meaning including but not limited to.

### Embodiment 1

### Acquisition of the E3 Ubiquitin Ligase Gene RING-Finger E3 Ligase 1 (RFEL1) Sequence

Through transcriptome sequencing analysis, one E3 ubiquitin ligase gene, TuG1812G0100001368 (named RFEL1), is obtained. Gene-specific primers are designed based on the coding sequence (CDS) of TuG1812G0100001368 from G1812 in the Triticum urartu database, and amplification is performed using complementary DNA (cDNA) samples from leaves of Triticum urartu material PI428322 to obtain the full-length sequence of RFEL1. The specific operating procedure is as follows: under room temperature conditions, Triticum urartu accession PI428322 (sourced from the Laboratory of Crop Genomics and Molecular Breeding, College of Agronomy, Henan Agricultural University) is planted in seedling pots. Leaves are collected from 10-day-old seedlings, and total ribonucleic acid (RNA) is extracted using Trizol reagent (Tri-Reagent), followed by reverse transcription using a First-strand cDNA Synthesis Mix. Then, using forward primer (FP): 5′-ATGGATGACAGTTGCGCG-3′ (SEQ ID NO. 1) and reverse primer (RF): 5′-CTATTTCTTTCCATTGCTGGAAAAG-3′ (SEQ ID NO. 2) as primers and PI428322 leaf cDNA as the template, polymerase chain reaction (PCR) amplification is performed using KOD-FX high-fidelity enzyme (KOD-FX Neo, TOYOBO) The amplification program is: pre-denaturation at 94 degrees Celsius (° C.) for 5 minutes (min); 35 cycles of denaturation at 98° C. for 20 seconds (s), annealing at 58° C. for 15 s, and extension at 68° C. for 3 min; followed by a final extension at 68° C. for 10 min. The reaction volume is 50 microliters (μL), consisting of 2 μL of cDNA, 1.5 μL each of the 10 micromolar (M) primers FP and RF, 10 μL of 2 millimolar (mM) dNTPs (deoxynucleotide triphosphates), 1 μL of KOD-FX Taq (TOYOBO), 25 μL of 2×KOD-FX PCR Buffer, and 9 μL of ddH₂O (double-distilled water). The full-length CDS sequence of RFEL1 is obtained, the specific sequence of which is as shown in SEQ ID NO. 3, and its encoded amino acid sequence is as shown in SEQ ID NO. 4.

SEQ ID NO. 3:
ATGGATGACAGTTGCGCGGTGTGCGCGGACGCGCTCGAATGGGTGGCTT
ACGGGCCGTGCGGGCACCGCGAGGTGTGTTCAACCTGCGTCGTCCGCCT
TCGCTTCGTACTCGAGGATTCTCTATGCTGCATCTGTAAGACGGATTGC
CCCTCCGTCTTCGTCACAAAGGCCATGGGAGATTACACAAAAGTGATCT
CGGATTTCTCTGTTTTGCCCACTGAGGCAAATGAGGGAAATGTGGGGGA
GTACTGGTACCATGAGGATACAAAGGCATACTTTGATGATGCTGATCAT
TATAAGATGATAAGGGCGATGTGCCGACTTTCTTGTAGTGTATGTGACA
AAGCTGAGGACCAGGTTGGTCAGGCAGCACAAGCAAAGCGCCGAAGCAG
GTTCAAGAGCATTGATCAGCTAAAGGGACATTTGTTCCATCAGCATAGG
TTATACATGTGTAATCTTTGCTTGGAGGGGAGAAAGGTATTCATTTGTG
AACAGAAGCTTTATACAAGGGCACAGTTAGCTCAGCATACAAAAACTGG
TGACTCTGAGGTGGATGGCTCTGAGATTGAACGCAGTGGTTTTGCAGGA
CACCCAGTGTGTGAATTTTGTAAATATCCATTATATGGAGATAATGAGC
TTTACACACATATGTCCAGAGAACACTATTCGTGCCACATATGTCAAAG
GCAGCATCCTGGGCAGTATGATTATTTCCGGAACTATGATGATTTAGAG
ATGCATTTTCGTAAAGATCATTTCCTCTGTGAAGATGATGCATGTTTGG
CCAAGAAATTTGTTGTCTTCCAGAGTGACGCAGAGATCAAGAGACATAA
TGCTATGGAGCATGGTGGGCGGATGTCTCGTGCCCAGAGGAATGCGGCA
CTTCAGATACCTACCAGTTTTATATACCAAAGGAATGAGCAAGATCAAA
GGCGTGGCAGAGGTAGGGGTCGTAATGCTCACCATGACAGACCTGACAG
GGATTTCTCATTACCTGTGCGGGATGGCAGTGCAACTGCAGACCATGGC
CTTGGAAGTCGAGTTGATAGTGTTGCAGGGCCTTtCcaGTCATTAAGTG
TCAGTTCCAGTTCTGGTCGAACAGAAACTGGTCGAAGCTTAGGGAATGG
TCGCTTGCTCGAGCAGTTGTCTTTTCCTCCACTTCAAGATCAGGATATT
CCTGATGCCAGGATGGATGCTGTTCCCTATGAAACCTCGTTTCCTCCCG
TCTCAGAGCAGCAATCAAGGTATGCGCTGGCTCTTAATCAGAGCTCAAG
GGGTTCTGCGAGGCTTGGTGATGAATCATTATTCCCTCCATTGCCTGGg
TCAAGTAACAAGGGTTCTGCTTCAACACAACAGGGGTTGCAAAGTCTTG
CTAAGAACACACTTGCATCAAGGCTGCAACAACGTAGTAAGGGCACCGT
GAAGGTACTATATTCTGCTCAGTCTCAAACAGCTGAAAATCCTGAGATT
GTACCTCATGTTTCCACTTCCACCCAGACATGGCCTACACCTGAGCAGG
GGTTACATCTCTCTGGTTCTTCTCAACTTTGGATTGTAACTCAATCAAC
AAGAGAAAATGGGCTCATGCCATCTGCCTCCAGCGGTTCAGCATGGAAT
TCCAGAGCTTCAAACAAGATGAAGCACTCTATTTCGACCCCTAATTTTG
TTTCTGGTGGGTCCTCTGCCCAGGCATCAAGTACAGCTTATGGCAATAA
AAAGCAACTGCCACCGCAAAGCAGCCAACCTTTGCCTGTTGTGGAGGAT
GTTCGGCAAGCAAACAAATCCCTTGTGGAAAGAATGCGTGTTGCATTAG
GAATGGATGAGGATAGGTTCTCTGCGTTCAAAGAAATTGCTAGTGAATA
CCGTCAAGGTGTCATTGATACTTCAGAGTATCTCTCGTATGTGGAGCAG
TTTGGTATATCACATCTTGTTCCTGAAATGGCTAAGTTGTTGCCTGATC
CTCTGAAGCAGATGGAACTTGCTGATGCCTATTACACCAaCATGCGTTT
TAGAAGTCTCCAAGAAAACGGCGGTTGTGGAACCATTACTGTGAAAGAG
AACAAGCGTAGAAATAAGGGGAAGGGAAAAACACCTGATGCAGAAACAG
CTCCTGCTAAGGATGCAAGTGAATCGCTAGCTGATAGCTTTATGGACAC
TGTAAGGAAGCTTCAGTTGAACAACAAGGCTCAAGAAGGAGAGGCTGCA
GTGCTTTCGAAGGATGGGTATCGATCTTCCAAGGAAAAAATCCCACTAT
CAGCTGGAGGGTCATCCTGTGGTACAAATATGGGTCTAGACGGTGATCC
AGTTGCCATTTCAAAGGCGTCTGGCACTAGTAGGTATGTGGGCAAGGGT
GGAGGAAGCACCGGTAGCAGCAGCAGTAACAACAAACAATCGAAGAAGA
CATCAAAGTTTCTCAGAGCTCGGTTGGGTGACAACTCATTGGCTACACT
TGATTTTAGTCATCCTGATGTGAGCCCTGAACGACCTGTAAGGGAGACA
CAAGTCCTGCAAACTGGGTTGCCTGTGCGAAGTGTTTGGAAGAATGGTG
CAGCACAGAAGCTCTTTTCCAGCAATGGAAAGAAATAG;
SEQ ID NO. 4:
MDDSCAVCADALEWVAYGPCGHREVCSTCVVRLRFVLEDSLCCICKTDC
PSVFVTKAMGDYTKVISDFSVLPTEANEGNVGEYWYHEDTKAYFDDADH
YKMIRAMCRLSCSVCDKAEDQVGQAAQAKRRSRFKSIDQLKGHLFHQHR
LYMCNLCLEGRKVFICEQKLYTRAQLAQHTKTGDSEVDGSEIERSGFAG
HPVCEFCKYPLYGDNELYTHMSREHYSCHICQRQHPGQYDYFRNYDDLE
MHFRKDHFLCEDDACLAKKFVVFQSDAEIKRHNAMEHGGRMSRAQRNAA
LQIPTSFIYQRNEQDQRRGRGRGRNAHHDRPDRDFSLPVRDGSATADHG
LGSRVDSVAGPFQSLSVSSSSGRTETGRSLGNGRLLEQLSFPPLQDQDI
PDARMDAVPYETSFPPVSEQQSRYALALNQSSRGSARLGDESLFPPLPG
SSNKGSASTQQGLQSLAKNTLASRLQQRSKGTVKVLYSAQSQTAENPEI
VPHVSTSTQTWPTPEQGLHLSGSSQLWIVTQSTRENGLMPSASSGSAWN
SRASNKMKHSISTPNFVSGGSSAQASSTAYGNKKQLPPQSSQPLPVVED
VRQANKSLVERMRVALGMDEDRFSAFKEIASEYRQGVIDTSEYLSYVEQ
FGISHLVPEMAKLLPDPLKQMELADAYYTNMRFRSLQENGGCGTITVKE
NKRRNKGKGKTPDAETAPAKDASESLADSFMDTVRKLQLNNKAQEGEAA
VLSKDGYRSSKEKIPLSAGGSSCGTNMGLDGDPVAISKASGTSRYVGKG
GGSTGSSSSNNKQSKKTSKFLRARLGDNSLATLDFSHPDVSPERPVRET
QVLQTGLPVRSVWKNGAAQKLFSSNGKK.

### Embodiment 2

### Functional Verification of the E3 Ubiquitin Ligase Gene RFEL1 in Wheat Resistance to Stripe Rust

The function of the E3 ubiquitin ligase gene RFEL1 in wheat resistance to stripe rust is verified through BSMV-VIGS (Barley stripe mosaic virus-induced gene silencing) and transgenic overexpression.

The function of the RFEL1 gene is first verified by VIGS assay. A 140 base pairs (bp) sequence (SEQ ID NO. 7) is reversely constructed into the RNA 7 chain of Barley stripe mosaic virus (BSMV) using primers RFEL1-VIGS (SEQ ID NO. 5 and SEQ ID NO. 6) to form the recombinant plasmid BSMV:RFEL1. The RFEL1 fragment is then introduced into wheat leaves via BSMV. At the wheat one-leaf-one-tiller stage, the recombinant viral vector BSMV:RFEL1 and the control viral vector BSMV:GFP are separately rub-inoculated onto the second leaf of 15 PI428322 plants each.

SEQ ID NO. 5:
GATTCTTCTTCCGTTGCTAGCGACTTTCTTGTAGTGTATGTGACAA;
SEQ ID NO. 6:
TTTTTTTTTTTTTTAGCTAGCAAGATTACACATGTATAACCTATGC;
SEQ ID NO. 7:
GACTTTCTTGTAGTGTATGTGACAAAGCTGAGGACCAGGTTGGTCAG
GCAGCACAAGCAAAGCGCCGAAGCAGGTTCAAGAGCATTGATCAGCT
AAAGGGACATTTGTTCCATCAGCATAGGTTATACATGTGTAATCTT.

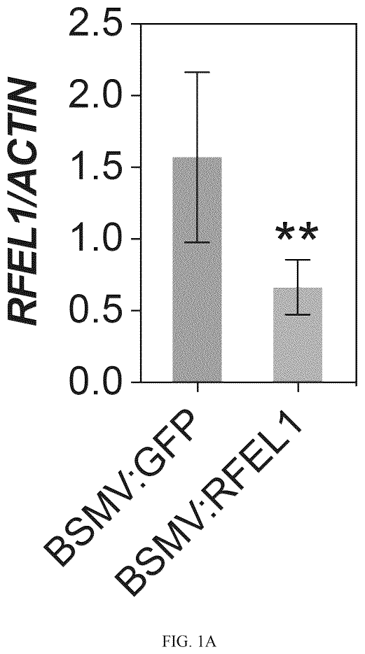

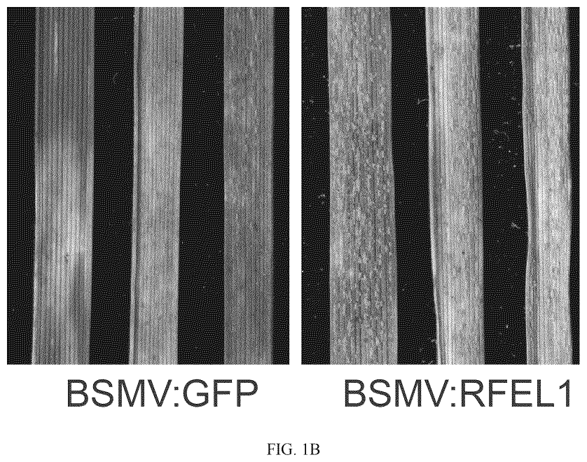

On day 14 after rub-inoculation with the virus, the fourth leaves are collected to extract RNA for detecting the silencing efficiency of the RFEL1 gene. Successfully silenced plants are inoculated with stripe rust fungus CYR34 (sourced from the Laboratory of Crop Genomics and Molecular Breeding, College of Agronomy, Henan Agricultural University). Disease resistance phenotypes are observed at 14 days post-inoculation, and fungal biomass is determined. The specific method for fungal biomass determination is as follows: infected leaves at 14 days post-inoculation are collected, DNA is extracted from infected leaves, and quantitative reverse transcription polymerase chain reaction (qRT-PCR) is performed (amplification primers: Triticum aestivum Elongation Factor 1-alpha (TaEF1a) FP: 5′-TGGTGTCATCAAGCCTGGTATGGT-3′ (SEQ ID NO. 8), RP1: ACTCATGGTGCATCTCAACGGACT (SEQ ID NO. 9); Puccinia striiformis Elongation Factor 1 (PsEF1) FP: 5′-TTCGCCGTCCGTGATATGAGACAA-3′ (SEQ ID NO. 10), RP2: 5′-ATGCGTATCATGGTGGTGGAGTGA-3′ (SEQ ID NO. 11). Amplification program: pre-denaturation at 95° C. for 30 s; followed by 40 cycles of denaturation at 95° C. for 10 s, annealing at 60° C. for 20 s, and extension at 72° C. for 20 s. The reaction volume is 10 μL, consisting of 1 μL of genomic DNA, 0.2 μL each of the 10 μM primers TaEF1a FP, RP1, PsEF1 FP, and RP2, and 5 μL of Taq SYBR•@Green qPCR premix (Lablead), and 3.6 μL of ddH₂O). The relative value of the stripe rust elongation factor (PsEF1) to the wheat elongation factor (TaEF1a) is used to determine the stripe rust biomass. The results show that transient silencing of the RFEL1 gene in PI428322 reduces its resistance to Pst CYR34 (FIG. 1A, FIG. 1B, and FIG. 1C).

The present disclosure further introduces the RFEL1 gene into the susceptible common wheat cultivar Fielder (sourced from the Laboratory of Crop Genomics and Molecular Breeding, College of Agronomy, Henan Agricultural University). Firstly, primers are designed based on the full-length CDS sequence of RFEL1, and the full-length CDS sequence of RFEL1 (SEQ ID NO. 3) is obtained by PCR amplification using PI428322 cDNA as the template. The full-length CDS sequence of RFEL1 is then ligated into the pCAMBIA1305 vector with a green fluorescent protein (GFP) tag driven by the ubiquitin (UBI) promoter. Using the RFEL1-GFP fusion vector as a template, specific primers, upstream primer SEQ ID NO. 12 and downstream primer SEQ ID NO. 13, are designed to construct the pLH5 overexpression vector.

SEQ ID NO. 12:
TTTCAAAGGCGTCTGGCACT;
SEQ ID NO. 13:
TGTAGCCAATGAGTTGTCACC.

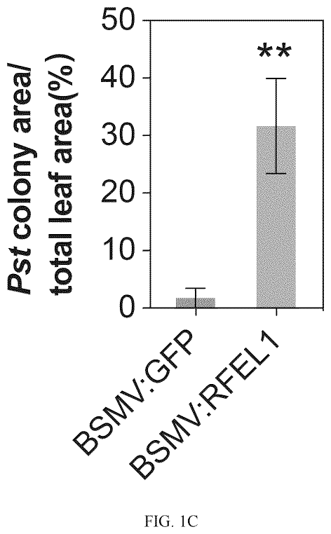

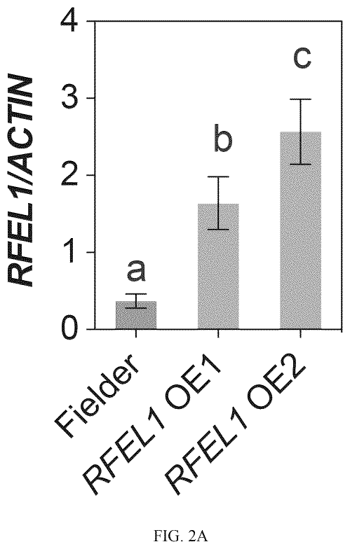

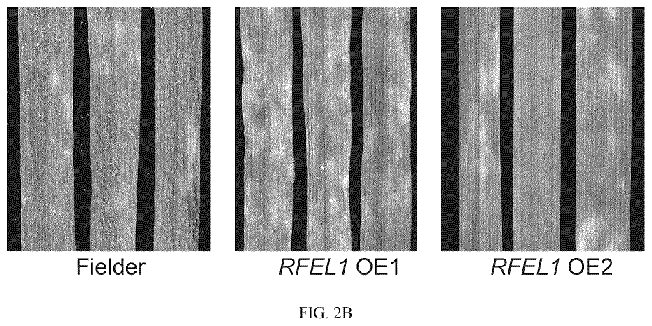

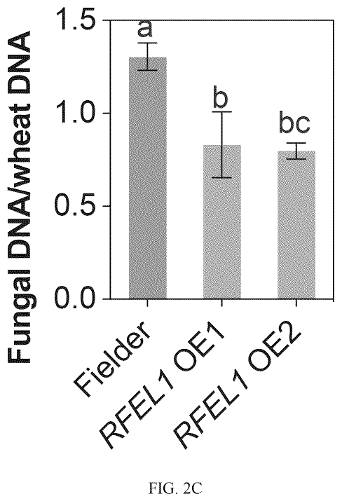

The pLH5 overexpression vector containing the RFEL1-GFP fusion protein is introduced into common wheat Fielder via Agrobacterium-mediated transformation to obtain stable transgenic plants. The presence of the RFEL1 gene in the transgenic plants is verified by qRT-PCR (reaction program: pre-denaturation at 95° C. for 30 s; 40 cycles of denaturation at 95° C. for 10 s, annealing at 60° C. for 20 s, and extension at 72° C. for 20 s. The reaction volume is 10 μL, consisting of 1 μL of cDNA, 0.2 μL each of the 10 μM forward primer and the 10 μM reverse primer, 5 μL of Taq SYBR•@Green qPCR premix (Lablead), and 3.6 μL of ddH₂O). This reaction is used to confirm the presence of the RFEL1 gene in the transgenic plants. Two stable T2 generation positive transgenic lines and the control Fielder are grown in a greenhouse under conditions of 16 hours (h) light and 8 h dark at 22° C. for 10 days, then inoculated with stripe rust fungus CYR34. Disease resistance phenotypes are observed and stripe rust biomass is measured at 14 days post-inoculation. The results show that RFEL1 transgenic plants of Fielder exhibit enhanced resistance to stripe rust fungus CYR34 (FIG. 2A, FIG. 2B, and FIG. 2C).

To verify the function of the E3 ubiquitin ligase gene RFEL1 in wheat resistance to multiple physiological races of stripe rust fungus, T2 generation positive transgenic lines and the control Fielder are grown in a greenhouse under conditions of 16 h light and 8 h dark at 22° C. for 10 days, then inoculated with stripe rust fungi CYR17, CYR32, and CYR33. Disease resistance phenotypes are observed at 14 days post-inoculation. The results show that RFEL1 transgenic plants of Fielder exhibit enhanced the resistance of Fielder to stripe rust fungi CYR17, CYR32, and CYR33 (FIG. 3).

### Embodiment 3

The role of the E3 ubiquitin ligase gene RFEL1 in broad-spectrum disease resistance in wheat

To verify the role of the E3 ubiquitin ligase gene RFEL1 in broad-spectrum disease resistance in wheat, RFEL1 transgenic plants are inoculated with powdery mildew fungus E09 (sourced from the Laboratory of Crop Genomics and Molecular Breeding, College of Agronomy, Henan Agricultural University) and leaf rust fungus Pt23a (sourced from the laboratory of Professor Zheng Wenming, College of Life Sciences, Henan Agricultural University) for disease resistance phenotype identification.

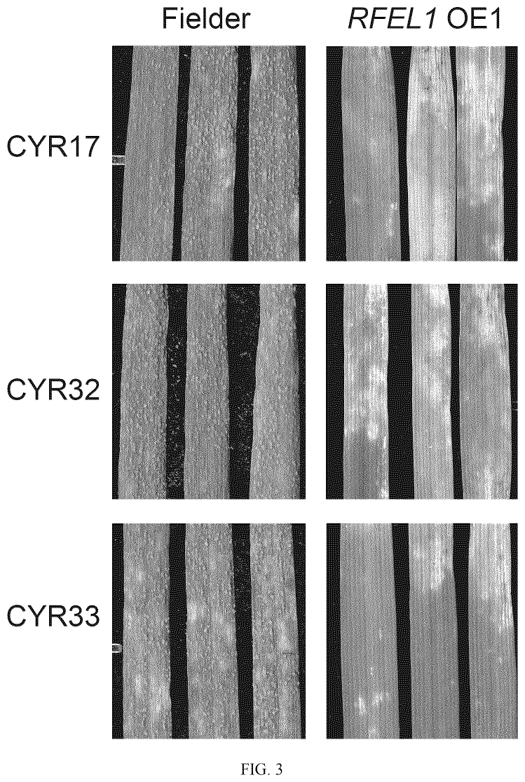

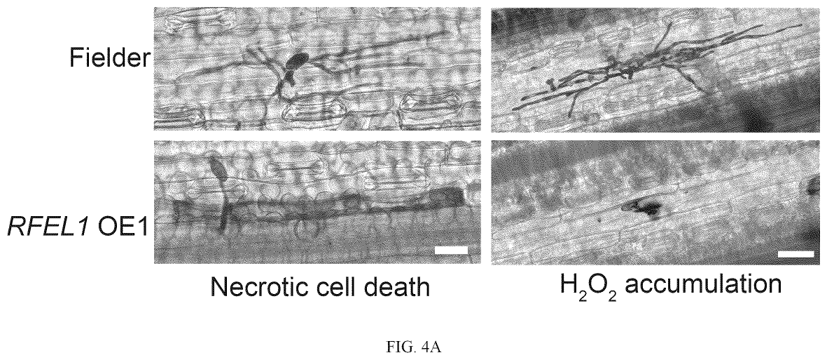

To verify the role of the E3 ubiquitin ligase gene RFEL1 in wheat resistance to powdery mildew, two stable T2 generation positive transgenic lines and the control Fielder are grown in a greenhouse under conditions of 16 h light and 8 h dark at 22° C. for 10 days, then inoculated with powdery mildew fungus E09. Samples are taken at 48 hours post-inoculation. The growth and development status of hyphae is observed using Coomassie Brilliant Blue staining; cell death is observed using trypan blue staining solution; and hydrogen peroxide (H₂O₂) accumulation is observed using 3,3′-diaminobenzidine (DAB) staining solution. The results show that compared to wild-type Fielder, RFEL1 transgenic plants exhibit slower hyphal growth of powdery mildew fungus E09, displaying larger areas of cell death and greater accumulation of hydrogen peroxide. Phenotypes observation at 7 days post-inoculation show that RFEL1 transgenic plant exhibit fewer spores on the leaf surfaces and enhanced disease-resistant (FIG. 4A and FIG. 4B).

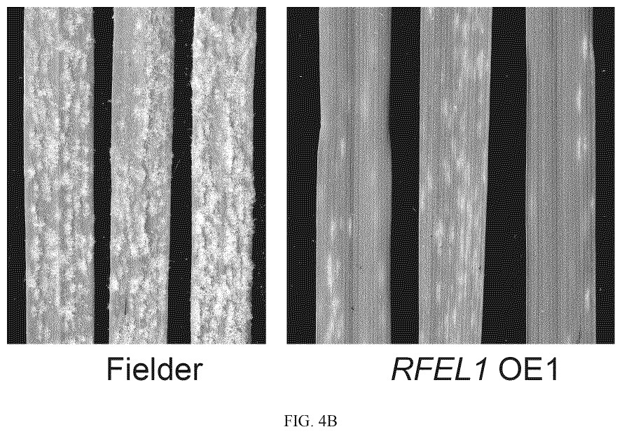

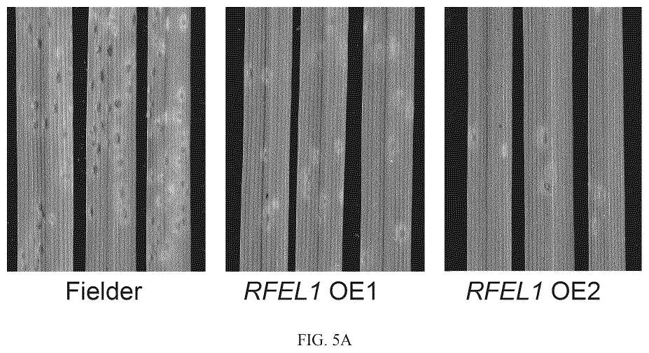

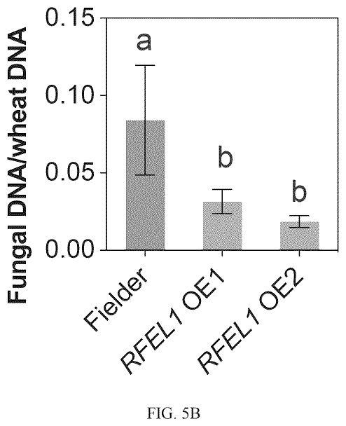

To verify the role of the E3 ubiquitin ligase gene RFEL1 in wheat resistance to leaf rust, two stable T2 generation positive transgenic lines and the control Fielder are grown in a greenhouse under conditions of 16 h light and 8 h dark at 22° C. for 10 days, then inoculated with leaf rust fungus Pt23a. Leaf rust biomass measurement and phenotype observation are performed at 14 days post-inoculation. The results show that compared to wild-type Fielder, RFEL1 transgenic plants exhibit stronger resistance to leaf rust fungus Pt23a (FIG. 5 A and FIG. 5B).

It needs be noted that the embodiments described above are only illustrative of the optional modes of the present disclosure and do not limit the scope of the present disclosure. Without departing from the design spirit of the present disclosure, various modifications and improvements made by those of ordinary skill in the art to the technical schemes of the present disclosure shall fall within the protection scope defined by the claims of the present disclosure.

What is claimed is:

1. A method for enhancing wheat resistance to stripe rust, powdery mildew, and leaf rust, comprising following steps:
step 1, constructing an overexpression vector using a wheat E3 ubiquitin ligase gene RING-finger E3 ligase 1 (RFEL1) as a target gene;
step 2, transforming a constructed overexpression vector into wheat; and
step 3, screening to obtain positive transgenic wheat;
wherein the nucleotide sequence of the wheat E3 ubiquitin ligase gene RFEL1 is as shown in SEQ ID NO. 3.
